(19) United States
(12) Patent Application Publication
(10) Pub. No.: US 2002/0182655 A1
(43) Pub. Date: Dec. 5, 2002

(54) Method for identifying ligands for G protein-coupled receptors

(76) Inventors: Evi Kostenis, Frankfurt am Main (DE); Johann Gassenhuber, Wiesbaden (DE); Kirsten Uhlenbrock, Frankfurt (DE)

Correspondence Address: FINNEGAN, HENDERSON, FARABOW,, GARRETT AND DUNNER, Franklin Square Bldg., Suite 700, 1300 I Street, N.W., Washington, DC, 20005-3315, US

(21) Appl. No.: 10/146,065

(22) Filed: May 16, 2002

(30) Foreign Application Priority Data
May 17, 2001 (DE) 10123958.0-41

(51) Int. Cl. G01N 33/567; C12N 1/18; C12N 5/06; C12N 5/08; C12N 15/85

(52) U.S. Cl. 435/7.21; 435/455; 435/358; 435/354; 435/367; 435/369; 435/254.2

(57) ABSTRACT

Methods for identifying a ligand of a G protein-coupled receptor (GPCR), such as ligands of orphan GPCRs, are provided. The methods include providing at least one cell containing a G-alpha protein that couples to at least one GPCR. The at least one cell is transformed with a recombinant DNA including a nucleic acid encoding a GPCR and the nucleic acid is expressed. At least one chemical compound is contacted with the transformed at least one cell and a calcium sensitive-fluorescent dye to form a first mixture. The at least one chemical compound is also contacted with at least one untransformed cell and the calcium sensitive-fluorescent dye to form a second mixture. Fluorescence from the first and second mixtures is measured and compared to determine whether the at least one chemical compound is a ligand of the GPCR. Nucleotide sequences, amino acid sequences, vectors, cells, and pharmaceuticals thereof are provided.

### CROSS-REFERENCE TO RELATED APPLICATION

The present application claims priority under 35 U.S.C. §119 of German Application No. 10123958.0-41, filed May 17, 2001, the disclosure of which is expressly incorporated by reference herein in its entirety.

### DESCRIPTION

1. Field of the Invention

The present invention relates to a method for identifying ligands for G protein-coupled receptors (“GPCRs”), such as orphan GPCRs, by means of a fluorometric imaging plate reader (“FLIPR”).

2. Background of the Invention

GPCRs play a central part in a multiplicity of physiological processes. It is assumed that in the human genome about 1,000 genes code for members of this receptor family. Approximately 60% of the pharmaceuticals presently available through prescription act as GPCR agonists or antagonists. This underlines the importance of this receptor class for the pharmaceutical industry. Owing to the size and importance of said protein family, and in view of the fact that physiological ligands are still unknown for many GPCRs (“orphan GPCRs”), this receptor class will be one of the most important reservoirs for suitable target proteins in the search for novel pharmaceuticals in the future.

GPCRs are a family of integral membrane proteins that are located on cell surfaces. They receive signals from extracellular signaling substances (e.g., hormones, neurotransmitters, peptides, lipids) and transfer these signals into the cell interior via a family of guanine nucleotide-binding proteins, the “G proteins”. Depending on the receptor specificity, the G protein activated, and the cell type, GPCRs activate various signal transduction pathways.

All GPCR polypeptide chains fold into seven α-helices that span across the phospholipid bilayer of the cell membrane. The seven membrane passages result in the formation of extra- and intracellular loops that allow extracellular ligand binding and intracellular coupling of G proteins. For this reason, GPCRs are also known as seven-pass transmembrane receptors.

All G protein-coupled receptors act according to a common mechanism: binding of an extracellular ligand leads to a conformational change in the receptor protein so that said receptor protein can contact a G protein. G protein-mediated signal transduction cascades in the cell finally lead to a biological response of the cell.

G proteins are heterotrimeric proteins that consist of subunits α, β and γ, and are located on the inside of the cell membrane via lipid anchors. Coupling of activated GPCRs to G proteins causes GDP/GTP exchange on the Gα subunit and dissociation of the heterotrimer into an α and a βγ subunit. Both the activated a subunit and the βγ complex can influence intracellular effector proteins.

Activation of membrane-bound adenylate cyclase (AC) by Gαs-type G proteins, for example, leads to an increase in the intracellular cAMP level or, in the case of activation by Gαi-type G proteins, to a decrease of the intracellular cAMP level. Gq-type G proteins activate phospholipase C (PLC), which catalyzes the formation of inositol 1,4,5-triphosphate (IP3) and diacylglycerol (DAG). These molecules in turn lead to the release of Ca²⁺ from intracellular stores or to activation of protein kinase C (PKC), with further effects in both cases.

Apart from the G protein types mentioned above (Gαi/s, Gq), there are numerous other types that are denoted G16, G12/13, etc. The large variety of G protein types reflects the large variety of very different GPCR functions.

Most GPCRs bind only to one type of Gα subunit, i.e., they are selective for a particular signal transduction pathway. This narrow specificity is a great hindrance for the purpose of developing a method by which chemical compounds that switch on GPCR-dependent signal transduction pathways are to be identified. In addition, only those signal transduction pathways giving quick and readily analyzable read outs are suitable, for example, for an industrial assay with high sample throughput (high throughput screening; “HTS”). The increase in the intracellular Ca²⁺ level due to Gq or G16 proteins meets this requirement.

In recent years, “promiscuous” G proteins have increasingly been constructed with the aim of functionally coupling as many GPCRs as possible to the Ca²⁺ pathway and thus making them accessible for HTS screening. Promiscuity means the nonselectivity of the G protein for a GPCR. It is possible by means of molecular biological and biochemical methods to prepare promiscuous G proteins from hybrid G proteins or by mutagenesis within the G16 family. Thus it is possible, for example, by fusion of the Gαi receptor recognition region to the Gαq effector activation region, to prepare a Gαq/i hybrid that receives signals from Gi-coupled receptors, but switches on the Gαq-PLC-β signal transduction pathway. A hybrid of this kind, in which the 5 C-terminal amino acids of Gαq had been replaced with the corresponding Gαi sequence (Gαqi5) was first described by Conklin et al., Nature 363, 274-276 (1993).

This “recoupling” of receptors has the advantage that the assay endpoint (increase in the intracellular Ca²⁺ concentration in comparison with adenylate cyclase inhibition) is more readily accessible through measurement methods and can be used in high throughput screening. The FLIPR (Molecular Devices) is an apparatus that typically measures intracellular Ca²⁺ levels in 96-well and 384-well formats.

Examples of orphan GPCRs are human GPRs 3, 6, and 12. GPR is another term for G protein-coupled receptor. The numbers refer to specific receptors. All three receptors were originally found to be strongly expressed in the central nervous system. Since then, however, GPR 3, 6, and 12 expression has been found in the peripheral vascular system (endothelial cells and smooth muscle cells). It can be assumed therefore that these receptors play an essential role in the physiology/pathophysiology of the endothelium and thus of the entire human vascular system. The development of hypertension, atherosclerosis, or other cardiovascular disorders could be associated with these receptors.

On the basis of sequence comparisons, high homology to GPCRs with lipid ligands was found. The sequence similarities suggest that GPR3, 6, and 12 could likewise be lipid receptors.

The sequence information for the three receptor genes is publicly accessible. The sequence accession numbers are L32831 for the human GPR 3 gene, L36150 for the human GPR 6 gene and U18548 for the human GPR 12 gene. The information is available, for example, via www.ncbi.nlm.nih.gov.

GPCR ligands are commonly identified in laboratory experiments by trial and error. This procedure often has the disadvantage of being time-consuming and random.

The identification of GPCR ligands is complicated by the mechanism of action of human GPR3. It is known from the literature that human GPR3 can stimulate αs-type G proteins in a constitutively active manner (Eggerickx et al., Biochem J.; 309(3): 837-843, 1995). It has not been possible, however, to clarify whether GPR3 is a genuine, permanently active GPCR or a factor in the cell culture medium causes the constitutive activity.

Sequence comparisons reveal a relationship between the GPR 3, 6, and 12 receptors and receptors binding lipid-like ligands. Moreover, lipids are typically found in the cell culture medium. For this reason, it seems reasonable to speculate that lipids present in the medium could cause receptor activation.

Receptors that are already maximally activated cannot be stimulated any further so that the cells were subjected to starvation (reduction in serum and thus lipid in the medium) to find potential stimulators. It should be possible to switch on starving receptors by exogenous addition of the activator.

Like human GPR3, there is no physiological ligand known for rat GPR3. Only a partial sequence of this receptor is present in publicly accessible databases (accession number: L32829). This leads to the disadvantage, for example for selectivity studies regarding pharmaceuticals, that it is not possible to use said receptor for profiling said pharmaceuticals.

### SUMMARY OF THE INVENTION

The present invention is directed to providing a rapid and simple method by means of which one or more ligands for a GPCR can be identified. The present invention may be combined with bioinformatics assay methods and specific searching for physiological ligands.

The present invention is directed to an attempt to connect GPCRs to a HTS-suitable Ca²⁺ pathway, after stimulation by potential lipid activators.

The present invention is also directed to providing a complete isolated nucleic acid encoding rat GPR3 gene.

In one aspect, the present invention is directed to a method for identifying a ligand of a G protein-coupled receptor (GPCR), comprising:

a) providing at least one cell containing a G-alpha protein that couples to at least one GPCR via the Gq/Ca²⁺ signaling pathway;

b) transforming the at least one cell with a recombinant DNA comprising a nucleic acid encoding a GPCR and expressing the nucleic acid;

c) providing at least one chemical compound;

d) contacting the at least one chemical compound with the transformed at least one cell and a calcium sensitive-fluorescent dye to form a first mixture;

e) measuring fluorescence from the calcium-sensitive fluorescent dye in the first mixture in a fluorometric imaging plate reader (FLIPR);

f) contacting the at least one chemical compound with at least one cell according to a) and the calcium sensitive-fluorescent dye to form a second mixture;

g) measuring fluorescence from the calcium-sensitive fluorescent dye in the second mixture in the FLIPR; and

h) comparing the fluorescence measurement from e) with the fluorescence measurement of g) to determine whether the at least one chemical compound is a ligand of the GPCR.

In a further aspect, the present invention is directed to a GPCR ligand that has been identified by the method.

In yet another aspect, the present invention is directed to a pharmaceutical composition comprising the ligand and at least one additive that stabilizes the ligand.

In still another aspect, the present invention is directed to a method of making a pharmaceutical, comprising first mixing the ligand with the at least one additive to form an initial pharmaceutical composition; processing the initial pharmaceutical composition to a final pharmaceutical composition; and then introducing the final pharmaceutical composition into containers, provided with an instruction leaflet, and packaging the containers.

In another aspect, the present invention is directed to a method of treating a disease, such as a cardiovascular disorder, comprising administering to a host in need of such treatment an effective amount of a pharmaceutical composition comprising the ligand so that the ligand binds to a GPCR that functions incorrectly in the disease.

In yet another aspect, the present invention is directed to an isolated and purified polynucleotide sequence encoding rat GPR3 comprising a nucleotide sequence according to SEQ ID NO 1.

In a further aspect, the present invention is directed to purified rat GPR3, comprising an amino acid sequence according to SEQ ID NO 2.

In another aspect, the present invention is directed to an expression vector comprising the polynucleotide sequence.

In still another aspect, the present invention is directed to a cell comprising the expression vector.

In another aspect, the present invention is directed to a cell preparation, comprising a cell that expresses a protein for rat GPR3 comprising an amino acid sequence according to SEQ ID NO 2, wherein the cell has been transformed by an expression vector comprising the polynucleotide sequence.

In one aspect, the at least one cell is chosen from HEK 293, CHO, COS, mouse 3T3, and HeLa cell. In addition, the at least one cell may be prepared by primary culture from an organ of a mammal. In certain embodiments, the cell is a yeast cell.

In another aspect, the G-alpha protein couples GPCRs of different specificity for ligands to the Gq/Ca²⁺-signaling pathway.

In yet another aspect, the GPCR is an orphan GPCR.

In still another aspect, the GPCR is chosen from mammalian GPR3, GPR6, and GPR12. The GPCR may be rat GPR3, or the GPCR may be chosen from human GPR3, GPR6, and GPR12.

In another aspect, the at least one chemical compound is chosen from a peptide, polysaccharide, fatty acid, polynucleotide, and organic molecule. The at least one chemical compound may have a molecular weight between about 0.1 and 25 kDa, or between about 0.1 and 10 kDa.

In another aspect, the at least one cell is cultured in medium containing about 1 to 10% (v/v) fetal calf serum and about 250 to 350 μM suramin.

In still another aspect, prior to providing the at least one chemical, the at least one chemical is determined to be a possible ligand by bioinformatics or a literature search.

In yet another aspect, the ligand is chosen from a protein, polysaccharide, fatty acid, and polynucleotide. The ligand of the method may be a ligand for GPR3, GPR6, or GPR12. In certain embodiments, the ligand is LPA, S1P, or suramin.

The invention also relates to a method for identifying a ligand of a G protein-coupled receptor (GPCR), which comprises:

a) providing a cell whose endogenous signal transduction cascades allow, e.g., coupling of the orphan GPCRs GPR3, 6 and 12, and also rGPR3 to the Ca²⁺ pathway. The cells (e.g., HEK293) may be cultured in medium containing 1% (v/v) FCS, and 300 μM suramin may be added to reduce the endogenous, lipid-induced background signal;

b) transfecting the cell line from a) with a recombinant GPCR construct such that said GPCR may be overexpressed in said cell line;

c) providing at least one chemical compound that is, e.g., considered a possible ligand, owing to theoretical preliminary considerations supported by bioinformatics and by literature searches (Eggerickx et al., Biochem J.; 309(3): 837-843, 1995);

d) contacting the chemical compound from c) with the cell line from b);

e) measuring fluorescence in an FLIPR assay by means of a calcium-sensitive fluorescent dye, after the contacting according to d); and

f) evaluating the results of the measurement by comparing the results of the fluorescence measurement from e) with the results of a fluorescence measurement after contacting a cell according to a) with a chemical compound according to c).

### DESCRIPTION OF THE INVENTION

The following description is for purposes of illustrative discussion of the various embodiments of the present invention.

Unless otherwise stated, a reference to a compound or component, includes the compound or component by itself, as well as in combination with other compounds or components, such as mixtures of compounds.

As an overview, the present invention relates to a method for identifying a ligand of a G protein-coupled receptor (GPCR), such as ligands of orphan GPCRs. The method includes providing a cell containing a G-alpha protein that couples at least one GPCR to the Gq/Ca²⁺ signaling pathway. The cell is transformed with a recombinant GPCR construct such that the GPCR is expressed in or on the cell line. At least one chemical compound is contacted with the transformed cell to form a first mixture. After contacting the at least one chemical compound with the cell, fluorescence of the first mixture from a calcium-sensitive fluorescent dye is measured in a fluorometric imaging plate reader (FLIPR). The fluorescence measurement is compared with a fluorescence measurement of a second mixture obtained by contacting an untransformed form of the cell with the same at least one chemical compound.

The GPCRs of the present invention (e.g., GPR3, 6, and 12) interact specifically with the Gαi class of G protein α subunits. Gαi activation causes adenylate cyclase inhibition. In addition, the βγ complex of the G proteins can cause intracellular release of Ca²⁺ from storage organelles. With regard to a FLIPR assay with Gαi- or αs-coupling GPCRs, G alpha proteins that couple GPCRs of different ligand specificity to the Ca²⁺ signaling pathway may be used. The “Gα16” protein is particularly suitable for carrying out the method of this invention. DE 10033353.2 discloses further Gα proteins that can be used for carrying out said method.

Recombinant techniques may be used to combine and assay different GPCRs in cell lines. The method may be carried out using a GPCR for which no physiological ligand is yet known. A physiological ligand is to be understood as meaning a molecule that is formed by an organism, in particular a mammal, which binds to said GPCR, activating a downstream G-alpha protein.

GPR 3, 6, or 12 are also suitable for carrying out the method of the invention. For example, the complete rat GPR3 receptor is suitable.

Cells that are suitable for use in the invention include those of mammals or yeast. These cells may be primary cells or cell lines. Examples of such cells are primary cells from mammalian organs (e.g., brain, muscle, fatty tissue, heart, lung, liver, kidney, blood vessels, hormonal glands, etc.). Suitable cell lines include, for example, CHO, HEK 293, COS, mouse 3T3, and HeLa cells.

“Providing” a cell may include its preparation, cultivation and further processing. Cells are provided, for example, by preparing suitable cell material from organs or tissues or by propagating suitable cell lines or microorganisms. Various suitable culture media can be used for cultivation. The cells are maintained at the optimum temperature for the organism. Where appropriate, preservatives, antibiotics, pH indicators, blood serum components, blood serum, auxiliaries or other substances are added to the growth medium used in each case. Processes for preparation, cultivation and further processing are described in standard textbooks (Example: Basic Cell Culture; Ed. J. M. Davis; IRL Press; 1994).

Recombinant techniques may be used to form a construct comprising a nucleic acid encoding a GPCR that is to be expressed in a cell. The polynucleotide sequence may be prepared by using known techniques, such as those described in F. M. Ausubel et al.; Current Protocols in Molecular Biology; John Wiley & Sons; New York; ISBN 0-471-50338-x.

For instance, a vector construct may be prepared by incorporating a polynucleotide coding for the amino acid sequence of a GPCR into an expression vector. An “expression vector” means a vector comprising a polynucleotide sequence that can be transfected into and expressed in a host cell. Expression vectors may be derived from plasmids, viruses, or cosmids and must be capable of autonomous replication. They generally contain an origin of replication, cleavage sites for restriction enzymes and marker genes such as, for example, antibiotic resistance genes. In an expression vector, the polynucleotide sequence that is to be propagated, or that has been introduced from the outside, may be under the functional control of a promoter. A promoter is a functional polynucleotide sequence of variable length that is used to control transcription, i.e., synthesis of mRNA of a polynucleotide sequence immediately 3′ of said promoter. There are promoters that are active only in prokaryotes, such as, for example, the lac, tac, and trc promoters, and also promoters that are active only in eukaryotes, such as, for example, CMV, T, and ADH promoters. Accordingly, prokaryotic expression vectors are different than eukaryotic expression vectors.

The recombinant vector construct, however, may comprise an expression vector usable in both eukaryotes and prokaryotes. The promoter may be inducible, by means of tryptophan, for example, or may be constitutively active. Examples of expression vectors are pUC18, pUC19, pBluescript, pcDNA3.1, etc.

“Transfection” means the introduction of foreign polynucletotide sequences into a host cell by means of a vector, and the subsequent propagation of said polynucleotide sequence to any number of identical copies within the host cell.

A cell line may be transfected with a recombinant construct by means of known methods, such as those described in the above-mentioned F. M. Ausubel et al.; Current Protocols in Molecular Biology, published by John Wiley & Sons, New York; ISBN 0-471-50338-x, or in Sambrook et al., A Laboratory Manual, Cold Spring Harbor Laboratory, ISBN 0-87969-309-6. Examples of such known methods are electroporation, Ca²⁺-phosphate coprecipitation and transfection with the aid of liposomes. Expression of transfected genes in the host cell may be detected by Western blotting of cell lysates of transfected cells. For this, too, the required laboratory protocols can be found in the manuals mentioned above. Specific antibodies for immunodetection of GPCR receptors that are suitable for carrying out the method of the invention (e.g., EDG 1-8) are commercially available. One of several suppliers is the company Exalpha Biologicals (www.exalpha.com). Rabbits may be immunized against GPR3, 6 and 12 with regard to producing specific antibodies.

A chemical compound may be provided by chemical synthesis or isolation of chemical substances from biological material. Biological material contains living or nonliving cells or components thereof.

Known methods may be used for chemical synthesis of the compound or isolation of a substance from cells. Such methods are available in textbooks such as Organic Synthesis Workbook; 1995; John Wiley & Sons; ISBN 3-527-30187-9, The Organic Chemistry of Drug Synthesis; 1998; John Wiley & Sons; ISBN 0-471-24510-0 or Bioactive Compounds from Natural Sources; 2001; Taylor & Francis; ISBN 0-7484-0890-8.

The compounds obtained by synthesis or isolation may be dissolved in a suitable solvent. Suitable solvents may contain water, buffers (e.g., Tris, HEPES, MOPS, etc.), monovalent and/or divalent ions (e.g., K⁺, Na⁺, Mg²⁺, Ca²⁺, etc.), acids (e.g., HCl, H₂SO₄, formic acid, acetic acid, etc.), bases (e.g., NaOH, etc.), alcohol (e.g., methanol, ethanol, glycerol), detergents (e.g., Na dodecyl sulfate, etc.), organic solvents (e.g., formamide, acetone, dimethyl sulfoxide, etc.) and other components, in particular solubilizers and stabilizers.

A chemical compound for the method of the invention could be a lipid, owing to the similarity of GPR3, 6, and 12 to lipid GPCRs. The chemical compound may be obtained from mammalian tissues or organs such as, for example, endothelial cell tissue, heart tissue, brain tissue, blood, serum or plasma. A compound suitable for carrying out the method of the invention may be a natural GPCR ligand.

The chemical compound may be contacted with said cell line by using known laboratory methods. Contacting may take place, for example, in Erlenmeyer vessels, tubes, Eppendorf vessels, or on microtiter plates. Temperature-controlled incubators, for which a constant temperature of, for example, 30° C. or 37° C. and constant CO₂ or humidity conditions can be set, may be used for said contacting. Contacting may in particular also be carried out in laboratory robot devices provided therefor (e.g., FLIPR). Contacting is possible for different periods of time, from a few seconds to minutes and up to several hours. The conditions to be chosen in each case depend on the receptor, the cell line and the chemical compound.

After contacting, fluorescence is measured by means of FLIPR. The system is suitable for measuring intracellular Ca²⁺ signals. Determinations may be carried out in microtiter plates having 96 or 384 wells. Binding of a ligand to a GPCR leads to intracellular release of Ca²⁺. The amount of Ca²⁺ released can be determined via a calcium-sensitive fluorescent dye (e.g., fluo-4). Comparison of the Ca²⁺ signal of a cell with the Ca²⁺ signal of a cell overexpressing GPR3, 6, or 12 makes it possible to determine a ligand for said GPCR. Such a ligand activates Ca²⁺ release much better in a cell that functionally overexpresses a GPCR. The technical equipment of the FLIPR system, including the reagents for determining Ca²⁺, is commercially available. One particular supplier is the company Molecular Devices with offices, inter alia, in Sunnyvale (Calif.), Ismaning (Germany) and Ashiya (Japan).

The invention also relates to ligands that are identified via the method described above. S1P and DHS1P cause Ca²⁺release in HEK293 cells.

Suramin may be used in the present assay to reduce the endogenous lipid background. For instance, experiments may be carried out in the presence of 300 μM suramin in order to reduce the background due to endogenous lipid receptor expression and to multiply the GPR 3, 6, and 12-mediated signals.

An organism, for example a vertebrate, forms a natural ligand in order to expediently activate a GPCR within the context of said organism. The purpose is in particular to initiate biochemical functions such as, for example, switching on action potentials in order to process sensory stimuli, switching on synthesis of a gene for a structural protein or a protein acting as messenger, releasing messengers, regulating metabolic functions, regulating organ functions such as heartbeat, blood pressure, or similar biological processes. A chemical compound that is suitable as a ligand for a GPCR binds to said GPCR. Ligand binding causes activation of said receptor. In the method of the invention, activation of a GPCR leads to the release of intracellular Ca²⁺ in the cell.

The invention furthermore relates to a pharmaceutical that comprises a ligand mentioned above and also additives for stabilizing said ligand and/or for formulating a pharmaceutical. Examples of additives include ionized and non-ionized tensids, phospholipids, dextran, glycerine, EDTA (ethylenediamine tetra-acetic acid), propylene glycol, triethanolamine, starch, and other compounds suitable for preparation of medicaments. The invention also relates to the preparation of a pharmaceutical. To this end, the above-mentioned ligand is mixed with the additives, and the pharmaceutical is then processed to the final form, introduced into containers, provided with an instruction leaflet and packaged.

The final form of a pharmaceutical relates to the final formulation, for example, as tablet, granules, spray, solution, ointment, tincture or other formulation forms. Processing to the final form refers to the preparation of the particular formulation.

The invention also relates to the use of a ligand, as mentioned above, for preparing a pharmaceutical suitable for treating a disease that is caused by incorrect functioning of the GPCR to which said ligand binds. For example, the ligand may be used for preparing a pharmaceutical for treating cardiovascular disorders and CNS disorders.

The ligand may be present together with an acceptable carrier in the form of a pharmaceutical composition. The carrier is acceptable to the extent that it is compatible with the other ingredients of the composition and is not harmful to the patient. The carrier may be a solid or a liquid or both. For instance, the carrier composition may compose a solid and a liquid phase, e.g., starch or another polymer suspended in water. The carrier may be formulated together with the compound as a single dose, for example, as a tablet that may contain from about 0.05% to 95% by weight of the active substance.

The pharmaceutical compositions of the invention may be produced according to any of the known pharmaceutical methods that essentially comprise mixing the components with pharmacologically acceptable carriers and/or excipients. Pharmaceutical compositions of the invention are those suitable for oral, rectal, topical, peroral (e.g., sublingual), and parenteral (e.g., subcutaneous, intramuscular, intradermal or intravenous) administrations.

The amount of a ligand as mentioned above required to produce a desired biological effect depends on a number of factors such as, for example, the specific compound chosen, the intended use, the type of administration and the clinical state of the patient. The amounts indicated below refer to a single ligand.

In general, the daily dose is in the range from about 0.3 mg to 100 mg (typically from about 3 mg to 50 mg) per day and per kilogram of body weight, for example about 3-10 mg/kg/day. An intravenous dose may be, for example, in the range from about 0.3 mg to 1.0 mg/kg and can most suitably be administered as an infusion of from about 10 ng to 100 ng per kilogram and per minute. Suitable infusion solutions for these purposes may contain, for example, from about 0.1 ng to 10 mg, typically from about 1 ng to 10 mg, per milliliter. Single doses may contain, for example, from about 1 mg to 10 g of the active substance. It is thus possible for ampoules for injections to contain, for example, from about 1 mg to 100 mg, and for single-dose formulations that can be administered orally, such as, for example, tablets or capsules, to contain, for example, from about 1.0 to 1000 mg, typically from about 10 to 600 mg.

The invention also relates to a polynucleotide sequence according to SEQ ID NO 1, which codes for a rat GPR 3. The invention furthermore comprises a protein for rat GPR 3, which comprises at least one amino acid sequence according to SEQ ID NO 2.

The invention also comprises an expression vector comprising the polynucleotide sequence of rat GPR 3 according to SEQ ID NO 1.

Moreover, the invention relates to a cell that expresses rat GPR 3 comprising at least the amino acid sequence according to SEQ ID NO 2, said cell having been transfected by means of an expression vector encoding rat GPR 3.

Regarding expression vectors and cells, reference is made to the explanations of the present invention that have been given in earlier sections. Methods for preparing and characterizing the polynucleotide sequence according to SEQ ID NO 1, the protein comprising at least the amino acid sequence according to SEQ ID NO 2, the expression vector and the cells, may be found in manuals such as F. M. Ausubel et al.; Current Protocols in Molecular Biology, John Wiley & Sons; ISBN 0-471-50338-x and Molecular Cloning; Cold Spring Harbor Laboratory; ISBN 0-87969-309-6.

The present invention will be further illustrated by way of the following Examples. These examples are non-limiting and do not restrict the scope of the invention.

### EXAMPLES

### Example 1

### Cloning of Human GPR 3, 6, and 12

The human genes of GPR 3, 6, and 12 contain no introns and can therefore be amplified from human genomic DNA by means of polymerase chain reaction (“PCR”). The coding sequences is cloned via the Hind III/Xbal sites of pcDNA 3.1 (Invitrogen). The cloning could take place in accordance with the steps of Example 2 as follows.

### Example 2

### Cloning of rat GPR 3

The sequence of the complete rat GPR 3 gene was propagated from rat brain cDNA by means of PCR amplification using the 5′ primer 5′-AAG CTT GCC ATG GCC TGG TTC TCA GCC GCC TCA-3′ (SEQ ID NO 3) and the 3′ primer (mouse) 5′-TCT AGA CTA GAC ATC ACT AGG GGA CCG GGA-3′ (SEQ ID NO 4). The 5′ primer additionally provides a Hind III site and generates a Kozak sequence in front of the start codon. The Kozak sequence provides a binding site for the mRNA and directs translation of RNA in eucaryotes. In the 3′ primer an Xbal site is also taken into account. The Xbal site in 3′ primer (SEQ ID NO 4) is TCTAGA.

The 990 bp amplification product was cloned into the Hind III/Xbal sites of pcDNA 3.1 (Invitrogen) and then sequenced. The resulting sequence is shown as SEQ ID NO 1.

### Example 3

### Expression of the GPR Genes

HEK293 cells (human embryonic kidney cell line) were cultured at 370° C. in DMEM supplemented with 10% (v/v) fetal calf serum, 10,000 IU/ml penicillin, 10,000 μg/ml streptomycin and 25 mM HEPES pH 7.0. CHO-K1 cells (Chinese hamster ovary cell line) were cultured at 37° C. in Iscove medium supplemented with 10% (v/v) fetal calf serum, 10,000 IU/ml penicillin, 10,000 μg/ml streptomycin, 1 mg/ml gentamycin, and 2 mM L-glutamine. Iscove medium is commercially available, for example, from Biochrom.

Transient transfections were carried out by incubating 5×10⁵ HEK293 cells or 2×10⁵ CHO-K1 cells in 6-well plates at 37° C. for 24 hours. The cells were then transfected with 1 to 2 μg of a vector, pcDNA 3.1, into which a GPR 3 gene was inserted by Hind III and Xba I sites, under the control of pcDNA 3.1. It is also expected that GPR 3, 6, or 12 may be cloned under control of the eukaryotic CMV promoter or of a control plasmid without corresponding cloned insert. The HEK293 cells were transfected with the aid of FuGene 6 transfection reagent (commercially available, for example from Roche Diagnostics) and the CHO cells were transfected with the aid of Lipofectamine reagent (commercially available, for example from GIBCO-BRL), in each case according to the manufacturer's instructions.

### Example 4

### Fluorometric Imaging Plate Reader (FLIPR) Assay

HEK293 cells were transferred to 96-well microtiter plates 24 hours after transient transfection. The cell density was 80,000 cells per well. The microtiter plates were coated with poly-D-lysine. The cells were kept in medium containing 1% (v/v) FCS (fetal calf serum) for 18 to 24 hours. After trypsinization, the cells were suspended in DMEM (Dulbecco's modified Eagle's medium) that also contained 25 mM HEPES (N-2-hydroxyethylpiperazine-N′-2-ethanesulfonic acid) pH 7.0, 1% (v/v) FCS, 10,000 IU/ml penicillin, 10,000 μg/ml streptomycin and 4 μM dye Fluo4 (fluorescent calcium indicator from Molecular Dynamics), and then incubated at 37° C. with 5% (v/v) CO₂ for 1 hour. The cells were then washed 3 times with PBS (phosphate-buffered saline) containing 1 mM MgCl₂, 1 mM EDTA and 0.4 mg/ml FAF-BSA (fatty acid-free bovine serum albumin). After the last washing step, the volume was 100 ,μl per cell. The compounds to be analyzed, i.e., S1P, DHS1P, suramin, and compounds from the lipid library, were present as 2 mM stock solutions in DMSO (dimethyl sulfoxide). These stock solutions were diluted 1:500 with the PBS solution containing 1 mM MgCl₂, 1 mM EDTA and 0.4 mg/ml FAF-BSA. Lipids were present in the form of triple-concentrated 900 μM solutions prior to the assay. Suramin (Sigma) was prepared in the form of a triple-concentrated 900 μM solution in PBS containing 1 mM MgCl₂, 1 EDTA, and 0.4 mg/ml FAF-BSA.

The FLIPR was programmed such that 50 μl of the triple-concentrated suramin stock solution were added to the cells, leading to a final suramin concentration of 300 μM. Fluorescence was measured in 3 second intervals for the first 3 minutes and in 10 second intervals for the last 2 minutes. Ca²⁺ signals of ligands were determined similarly.

The fluorescence data of the 18 to 36 second time interval were used to determine agonist activity.

The apparatus itself internally compares the zero values.

### Example 5

### Identification of Ligands for GPCR 3, 6, and 12

GPCR 3, 6, and 12 are orphan receptors of the GPCR protein family. It was possible with the aid of gene bank searches (EMBL) to identify the lipid receptors EDG (endothelial differentiation gene) GPCR and cannabinoid GPCR as the structurally closest relatives of GPR 3, 6, and 12. Homology of coding region within a single species is 65 to 68% with respect to the nucleotide sequence of GPR 3, 6 and 12. The human sequences show homologies of 87% (GPR 3), 83% (GPR 6) and 88% (GPR 12), respectively, in comparison with the rat sequences by means of FASTA and BLAST with ktup default being 2 for proteins and 6 for DNA.

Reverse transcriptase polymerase chain reaction (“RT-PCR”) analyses show matching expression of hGPR 3, 6, and 12 in cerebral tissues and cardiovascular organs (e.g., heart, kidney). On the basis of this result, it was possible to confirm expression in isolated endothelial cells and smooth muscle cells.

It was possible with the aid of a Ca²⁺ FLIPR assay in HEK293 cells to identify the lipids sphingosine 1-phosphate (S1P) and dihydrosphingosine 1-phosphate (DHS1P)as physiological ligands of hGPR 3, 6, and 12. Regarding the FLIPR assay, HEK293 cells were transferred to 96-well microtiter plates 24 hours after transient transfection. The cell density was 80,000 cells per well. The microtiter plates were coated with poly-D-lysine. The cells were kept in medium containing 1% (v/v) FCS (fetal calf serum) for 18 to 24 hours. After trypsinization, the cells were suspended in 100 μl DMEM (Dulbecco's modified Eagle's medium) that also contained 25 mM HEPES (N-2-hydroxyethylpiperazine-N'-2-ethanesulfonic acid) pH 7.0, 1% (v/v) FCS, 10,000 lU/ml penicillin, 10,000 μg/ml streptomycin and 4 μM dye Fluo4 (fluorescent calcium indicator from Molecular Dynamics), and then incubated at 37° C. with 5% (v/v) CO₂ for 1 hour. The cells were then washed 3 times with PBS (phosphate-buffered saline) containing 1 mM MgCl₂, 1 mM EDTA and 0.4 mg/ml FAF-BSA (fatty acid-free bovine serum albumin). After the last washing step, the volume was 100 μl per cell. Compounds to be analyzed, e.g., S1P and DHS1 P, were added at this stage. Fluorescence was measured by means of a fluorometric imaging plate reader in 3 second intervals for the first 3 minutes and in 10 second intervals for the last 2 minutes. The fluorescence data of the 18 to 36 second time interval were used to determine agonist activity. The reader itself equalizes internally the zero values.

Routine methods indicated that the EC₅₀ values for S1P and DHS1P were below 100 nM. Using a lipid library of 200 bioactive lipids at 300 μM produced no further ligands and, in addition, showed the ineffectiveness of cannabinoids.

In HEK293 cells, GPR 3, 6, and 12 are coupled to the Ca²⁺ signal transduction pathway without cotransfection of G₁₆ or other chimeric promiscuous Gαsubunits. The S1P/DHS1P-induced release of Ca²⁺ in HEK293 cells is pertussis toxin-sensitive, i.e., the signal transduction cascade runs via Gαi-type G proteins. Moreover, Ca²⁺ release is partly sensitive to sphingosine kinase inhibitors.

Orphan receptors GPR 3, 6, and 12 could be activated by natural ligands S1P and/or DHS1P. Both S1P and DHS1P are known already for triggering the GPCRs of the EDG family. The GPR 3, 6, and 12 as well as EDG receptors exhibit comparable expression patterns with respect to organic specificity. This is assumed as hind that both receptor classes might be involved in exerting comparable functions as apoptosis, angiogenesis, cell proliferation, platelet activation, vasoactivity, chemotaxis, and cell differentiation.

While the invention has been described in connection with certain preferred embodiments so that aspects thereof may be more fully understood and appreciated, it is not intended to limit the invention to these particular embodiments. On the contrary, it is intended to cover all alternatives, modifications and equivalents as may be included within the scope of the invention as defined by the appended claims.

What is claimed is:

1. A method for identifying a ligand of a G protein-coupled receptor (GPCR), comprising:
a) providing at least one cell containing a G-alpha protein that couples to at least one GPCR via the Gq/Ca²⁺ signaling pathway;
b) transforming the at least one cell with a recombinant DNA comprising a nucleic acid encoding a GPCR and expressing the nucleic acid;
c) providing at least one chemical compound;
d) contacting the at least one chemical compound with the transformed at least one cell and a calcium sensitive-fluorescent dye to form a first mixture;
e) measuring fluorescence from the calcium-sensitive fluorescent dye in the first mixture in a fluorometric imaging plate reader (FLIPR);
f) contacting the at least one chemical compound with at least one cell in accordance with a) and calcium sensitive-fluorescent dye to form a second mixture;
g) measuring fluorescence from the calcium-sensitive fluorescent dye in the second mixture in the FLIPR; and
h) comparing the fluorescence measurement from e) with the fluorescence measurement of g) to determine whether the at least one chemical compound is a ligand of the GPCR.

2. The method of claim 1, wherein the at least one cell is a HEK 293, CHO, COS, mouse 3T3, HeLa, or yeast cell.

3. The method of claim 1, wherein the at least one cell is from a primary culture of a mammalian organ.

4. The method of claim 1, wherein the G-alpha protein couples GPCRs of different specificity for ligands to the Gq/Ca²⁺-signaling pathway.

5. The method of claim 1, wherein the GPCR is an orphan GPCR.

6. The method of claim 1, wherein the GPCR is mammalian GPR3, GPR6, or GPR12.

7. The method of claim 5, wherein the GPCR is rat GPR3.

8. The method of claim 1, wherein the GPCR is human GPR3, GPR6, or GPR12.

9. The method of claim 1, wherein the at least one chemical compound is a peptide, a polysaccharide, a fatty acid, a polynucleotide, or an organic molecule.

10. The method of claim 1, wherein the at least one chemical compound has a molecular weight between about 0.1 and 25 kDa.

11. The method of claim 10, wherein the molecular weight is between about 0.1 and 10 kDa.

12. The method of claim 1, wherein the at least one cell is cultured in medium containing about 1 to 10% (v/v) fetal calf serum and about 250 to 350 μM suramin.

13. The method of claim 1, wherein prior to providing the at least one chemical, the at least one chemical is determined to be a possible ligand by bioinformatics or a literature search.

14. A GPCR ligand identified by the method of claim 1.

15. The GPCR ligand of claim 14, wherein the ligand has a molecular weight of between about 0.1 and 25 kDa.

16. The GPCR ligand of claim 14, wherein the ligand is a protein, a polysaccharide, a fatty acid, or a polynucleotide.

17. The GPCR ligand of claim 14, wherein the ligand is a ligand for GPR3, GPR6, or GPR12.

18. The GPCR ligand of claim 14, wherein the ligand is LPA, S1P, or suramin.

19. A pharmaceutical composition comprising:
the ligand of claim 14 and
at least one additive that stabilizes the ligand.

20. A method of making the pharmaceutical composition of claim 19, comprising:
first mixing the ligand with the at least one additive to form an initial pharmaceutical composition;
processing the initial pharmaceutical composition to a final pharmaceutical composition; and
then introducing and packaging the final pharmaceutical composition in containers provided with an instruction leaflet.

21. A method of treating a disease, comprising administering to a host in need of such treatment an effective amount of a pharmaceutical composition comprising the ligand of claim 14 so that the ligand binds to a GPCR that functions incorrectly in the disease.

22. The method of claim 21, wherein the disease is a cardiovascular disorder.

23. An isolated polynucleotide sequence encoding rat GPR3 comprising a nucleotide sequence according to SEQ ID NO 1.

24. Purified rat GPR3 comprising an amino acid sequence according to SEQ ID NO 2.

25. An expression vector comprising the polynucleotide sequence of claim 23.

26. A cell comprising the expression vector of claim 25.

27. A cell preparation, comprising a cell that expresses rat GPR3 comprising an amino acid sequence according to SEQ ID NO 2, wherein the cell has been transformed by an expression vector comprising the polynucleotide sequence of claim 23.


SEQUENCE LISTING (4 sequences)

SEQ ID NO: 1 - DNA, 966 bp, Rattus norvegicus
atggcctggttctcagccggctcaggcagtgtgaatgtgagcatagacccagcagaggaacctacaggcccagctacactgctgccctctcccagggcctgggatgtggtgctgtgcatctcaggcaccctggtgtcctgcgagaatgctctggtggtggccatcattgtgggcacgcctgccttccgcgcccccatgttcctgctggtgggcagcttggccgtagcagacctgctggcaggcctgggcctggtcctgcacttcgctgctgacttctgtattggctcaccagagatgagcttggtgctggttggcgtgctagcaacggcctttactgccagcatcggcagcctgctggccatcaccgttgaccgctacctttccctgtacaacgccctcacctactactcagagacaacagtaactcgaacctacgtgatgctggccttggtgtgggtgggtgccctgggcctggggctggttcccgtgctggcctggaactgccgggatggcctgaccacgtgcggcgtagtctatccactctccaagaaccatctggtggttctggccatcgtcttcttcatggtgtttggcatcatgctacagctctatgcccagatctgccgcattgtttgccgccacgcccagcagatcgccctccaacgacacctgctgcctgcctcccactacgtggccacccgcaagggcatcgccacattggccgtggtgcttggcgcctttgccgcctgttggttgcccttcactgtctactgcctcctgggagacgccaactctcctcctctctacacctaccttaccctgctccctgccacttacaactccatgatcaacccggtcatttacgccttccgcaaccaagacgtgcagaaggtgctgtgggccatctgctgctgctgttctacttccaagatcccattccggtcccggtcccctagtgatgtctag

SEQ ID NO: 2 - protein, 321 aa, Rattus norvegicus
MAWFSAGSGSVNVSIDPAEEPTGPATLLPSPRAWDVVLCISGTLVSCENALVVAIIVGTPAFRAPMFLLVGSLAVADLLAGLGLVLHFAADFCIGSPEMSLVLVGVLATAFTASIGSLLAITVDRYLSLYNALTYYSETTVTRTYVMLALVWVGALGLGLVPVLAWNCRDGLTTCGVVYPLSKNHLVVLAIVFFMVFGIMLQLYAQICRIVCRHAQQIALQRHLLPASHYVATRKGIATLAVVLGAFAACWLPFTVYCLLGDANSPPLYTYLTLLPATYNSMINPVIYAFRNQDVQKVLWAICCCCSTSKIPFRSRSPSDV

SEQ ID NO: 3 - DNA, 33 bp, Rattus norvegicus
aagcttgccatggcctggttctcagccgcctca

SEQ ID NO: 4 - DNA, 30 bp, Mus musculus
tctagactagacatcactaggggaccggga